(19) United States
(12) Patent Application Publication
(10) Pub. No.: US 2026/0224307 A1
(43) Pub. Date: Aug. 6, 2026

(54) DEVICES FOR CONTROLLING AN ENDOVASCULAR SYSTEM

(71) Applicant: UAB Inovatyvi Medicina, Kaunas (LT)

(72) Inventors: Vilius DAMBRAUSKAS, Kaunas (LT); Evaldas KALVAITIS, Kaunas (LT); Vaidas LABUNSKAS, Kaunas (LT); Eividas KAVOLELIS, Kaunas (LT); Edvardas SATKAUSKAS, Kaunas (LT)

(73) Assignee: UAB Inovatyvi Medicina, Kaunas (LT)

(21) Appl. No.: 19/159,581

(22) PCT Filed: Mar. 27, 2024
(86) PCT No.: PCT/EP2024/058195

§ 371 Date: Aug. 25, 2025

(30) Foreign Application Priority Data
Apr. 28, 2023 (DE) 102023111153.1

(51) Int. Cl. A61B 34/30

(52) U.S. Cl. CPC A61B 34/30; A61B 2034/301

(57) ABSTRACT

A method for inserting a first elongated medical member into a second elongated medical member, the method comprising: gripping, by a first and a second gripper, the first and second elongated medical members at respective first and second gripping points, wherein the first gripping point is a first portion of the first elongated medical member grippable by the first gripper and the second gripping point is a first portion of the second elongated medical member grippable by the second gripper; moving, by a first and second mover, the first and second grippers away from each other, wherein the first mover moves the first gripper from a first position to a second position and the second mover moves the second gripper from a third position to a fourth position; releasing the first and second grippers; gripping, by a third and a fourth gripper, the first and second elongated medical members at respective third and fourth gripping points, wherein the third gripping point is a second portion of the first elongated medical member grippable by the third gripper and the fourth gripping point is a second portion of the second elongated medical member grippable by the fourth gripper, wherein the first and second portions of the first and second elongated medical members are different respective portions; moving, by the first and second movers, the first and second grippers towards each other, wherein the first mover moves the first gripper from the second position to the first position and the second mover moves the second gripper from the fourth position to the third position; and releasing the third and fourth grippers.

### FIELD OF THE INVENTION

The present invention generally relates to methods for inserting a first elongated medical member into a second elongated medical member, and for extracting a first elongated medical member from within a second elongated medical member. Furthermore, the present invention generally relates to a medical apparatus for the insertion of a first elongated medical member into a second elongated medical member and/or the extraction of the first elongated medical member from within the second elongated medical member.

### BACKGROUND TO THE INVENTION

Endovascular specialists (for example (endo-)vascular surgeons, (interventional) cardiologists, (interventional) radiologists etc.) train, practice and develop intuitive skills to handle surgical tools. The mental imagery of skills of physicians also evolves by correlating their actions and responses of surgical tools within the human anatomy. However, there is a need for the insertion of a first instrument into a second instrument, and for the extraction of the first instrument from the second instrument in a more robotic manner. This is important should instruments need to be replaced before, during, or after a procedure.

The inventors have realized that existing vascular systems require a manual insertion/extraction. This can be difficult if the insertion or extraction is taking place during a procedure, especially where the tip of the second instrument should be kept in place during the insertion or extraction to avoid complications during the procedure.

There is therefore a need for improvements of (endo-) vascular systems, in particular when inserting a first instrument into a second instrument, or extracting the first instrument from the second instrument.

### SUMMARY OF THE INVENTION

The invention is set out in the independent claims. Preferred embodiments of the invention are set out in the dependent claims.

According to a first aspect, we describe a method for inserting a first elongated medical member into a second elongated medical member, the method comprising: gripping, by a first and a second gripper, the first and second elongated medical members at respective first and second gripping points, wherein the first gripping point is a first portion of the first elongated medical member grippable by the first gripper and the second gripping point is a first portion of the second elongated medical member grippable by the second gripper; moving, by a first and second mover, the first and second grippers away from each other, wherein the first mover moves the first gripper from a first position to a second position and the second mover moves the second gripper from a third position to a fourth position; releasing the first and second grippers; gripping, by a third and a fourth gripper, the first and second elongated medical members at respective third and fourth gripping points, wherein the third gripping point is a second portion of the first elongated medical member grippable by the third gripper and the fourth gripping point is a second portion of the second elongated medical member grippable by the fourth gripper, wherein the first and second portions of the first and second elongated medical members are different respective portions; moving, by the first and second movers, the first and second grippers towards each other, wherein the first mover moves the first gripper from the second position to the first position and the second mover moves the second gripper from the fourth position to the third position; and releasing the third and fourth grippers.

The first and second elongated medical members may be any suitable members such as, for example, a catheter, a guidewire, a stent, a stent balloon, a catheter balloon or any other elongated medical member. In particular, the first and/or second elongated medical member may be a medical member suitable for endovascular procedures.

The above method may also allow for a compact apparatus that undertakes the method. For example, should the first and second elongated medical members be 120 cm in length each, a normal system would need to be at least 240 cm in length.

With this method, the overall length of the apparatus/system can be greatly reduced, thereby compacting and reducing the size of the apparatus/system using the method.

The first and second grippers may be any suitable grippers that allow for the first and second elongated medical members to be gripped at respective first and second gripping points. The first and/or second grippers may be a mechanical gripper, a magnetic gripper, or any other suitable type of gripper. In particular, the first and/or second grippers may comprise a first gripping component configured to contact at least a first portion of an elongated member on a first side of said elongated member, a second gripping component configured to contact at least the first portion of said elongated member on a second side of said elongated member, wherein the first side is different from the second side, a guide configured to guide, during a movement of the second gripping component, at least a first portion of the second gripping component and an actuating component coupled to the second gripping component, wherein the second gripping component may be moveable between a first position and a second position based on an actuating force provided to the second gripping component via the actuating component, and wherein at least the first portion of the second gripping component may be guideable by the guide during movement of the second gripping component between the first position and the second position. The first gripping component may be configured to stay stationary or substantially stationary with respect to the guide. The second gripping component may comprise a first surface opposite to a first surface of the first gripping component. When the second gripping component is in the first position, said elongated member may be grippable between the first surface of the first gripping component and the first surface of the second gripping component. When the second gripping component is in the second position, said elongated member may not grippable between the first surface of the first gripping component and the first surface of the second gripping component. The first and second grippers may allow for the first and/or second elongated medical members to be held in place during the process, thereby reducing the likelihood of accidental movement of the members, thereby leading to a safer method. Additionally or alternatively to the above, the first and/or second grippers may be a spiral-type gripper that expands and contracts to release and grip a respective elongated medical member and/or a gripper that comprises two parts that move away from, and towards, one another to release and grip a respective elongated medical member and/or a torque gripper and/or a Tuohy borst valve/adapter and/or a hemostatic valve. A gripper is defined herein as a person or a thing that grips. That is to say, the gripper may, in some examples, replicate the gripping actions and/or gripping functionality of at least one human finger.

The first and second movers may allow for the first and/or second grippers to be moved. This may allow for the first and/or second elongated medical members to be gripped at different points of said members, thereby allowing for the members to be gripped at the optimal location. This may allow for a more efficient insertion of the first elongated medical member into the second elongated medical member and for a longer lifespan of the members as the respective grippers may not impact parts of the members during the insertion. A mover is defined herein as a person or thing that moves.

The third and fourth grippers may be substantially the same, or the same, as the first and second grippers. Additionally or alternatively to the above, the grippers may be a spiral-type gripper that expands and contracts to release and grip a respective elongated medical member and/or a gripper that comprises two parts that move away from, and towards, one another to release and grip a respective elongated medical member and/or a torque gripper and/or a Tuohy borst valve/adapter and/or a hemostatic valve.

The repeating of the method may allow for the first elongated medical member to be sufficiently inserted into the second elongated medical member, thereby allowing for the first elongated medical member to be safely stored within the second elongated medical member.

In some examples, a first pair of grippers comprises the first and third grippers, and a second pair of grippers comprises the second and fourth grippers. This may mean that the first and third grippers, and the second and fourth grippers, work as a tandem, and are dependent on each other. The actuation of each of the grippers may be controlled by a controller, with this controller also controlling the functioning of at least one of the pairs of grippers. The controller may, in some examples, be an embedded printed circuit board, PCB, controller. This controller may be placed on the same apparatus that undertakes the method. This controller may also function as a gateway/forwarder to another controller that is part of the system/apparatus, or is a controller external to the apparatus/system. A user may press a button, or other actuatable object, located on the system/apparatus which may then lead to a command to be given to the controller to start the actuation of each of the grippers. This user input can be an indication to start the insertion process according to the first aspect, an indication to start the retraction process, as described in relation to the second aspect, or any other suitable indication if a plurality of elongated medical members are inserted/extracted. The user input may, in some examples, be made from a remote location via a digital communication channel with the controller. The digital communication channel may use any known communication protocol.

In some examples, a first distance between the first and second positions, and a second distance between the third and fourth positions, are the same. This may allow for the first elongated medical member to be inserted into the second elongated medical member in a safe manner. This may be because the movement of the first and second gripping being equal means that the effective movement of the second elongated medical member during the insertion method counteracts the movement of the first elongated medical member, thereby meaning that the first member can be inserted into the second member in an apparatus that is compact, as mentioned above. This may also allow for a distal tip of the second elongated medical member to be kept in place during the insertion process. This may be very important should the insertion process take place during a procedure as unintended movements of the distal tip of an elongated medical member could jeopardize the safety of a patient. Throughout the disclosure, it is to be understood that the term “distal tip” may relate to a tip of a respective elongated medical member located distal from the site where the insertion and/or the extraction is occurring. That is to say the insertion and/or extraction occurs at, for example, a proximal end/tip of the second elongated medical member.

In some examples, a first distance between the first and second positions, and a second distance between the third and fourth positions, are proportional to one another. This may allow for the first elongated medical member to be inserted into the second elongated medical member in a safe manner. This may be because the movement of the first and second gripping being equal means that the effective movement of the second elongated medical member during the insertion method counteracts the movement of the first elongated medical member, thereby meaning that the first member can be inserted into the second member in an apparatus that is compact, as mentioned above. This may be very important should the insertion process take place during a procedure as unintended movements of the distal tip of an elongated medical member could jeopardize the safety of a patient.

In some examples, the first and third grippers, and the second and fourth grippers, are comprised in respective modules. This may allow for the grippers to be protected from external factors, thereby extending their lifetimes. This may also allow for it to be easier to undertake the method as the pairs of grippers are isolated from one another, thereby reducing the likelihood of unintended physical interaction between them during the moving steps of the method. This may also allow for the pairs to be placed at different predetermined distances from each other according to the elongated medical members, thereby allowing for a customizable apparatus/system, and indeed method, as the modules and method can be used for any suitable elongated medical members. This may also allow for maintenance to be undertaken in an easier manner as the pairs of grippers are located near each other.

In some examples, the predetermined distance is determined by a measurement sensor and/or scanning a code on the first elongated medical member, wherein the code comprises information on a length of the first elongated medical member. In some examples, the distance can be calculated via an encoder, or any other suitable method, coupled to the first and/or second movers. This may mean that the system which uses the method measures the distance of insertion. Additionally or alternatively, a user of the system undertaking the method may manually enter the member length into the system and/or scan a barcode associated with the first and/or second elongated medical member. This may mean that the system can be preprogrammed to ensure the end tip of the instrument under the change procedure is never more a second predetermined distance away from the front, i.e. patient end, of the device using the method, or is always at particular place. One, or both, of these methods may be controlled by a controller. This controller may be the same controller as described above, or a separate second controller. In some examples, there may be a controller coupled to each gripper, or each pair of grippers. This controller may be responsible for giving an action to the gripper, receive input from the PCB controller mentioned above, in order to control only that gripper/pair of grippers as an independent module. In some examples, there is a module/unit controller on the apparatus/system, which is configured to control the grippers via the controller mentioned above controllers. As an example, in the present disclosure, there would be one main controller and 4 sub-controllers, where each sub-controller controls a gripper. However, there skilled person understands that there may be any number of main controllers or sub-controllers. In other words, the moving and gripping steps of the method are undertaken by at least one controller coupled to each respective gripper.

In some examples, during the method, a distal tip of the second elongated medical member is maintained in a fifth position. This may be very important should the insertion process take place during a procedure as unintended movements of the distal tip of an elongated medical member could jeopardize the safety of a patient.

In some examples, said method is undertaken by a controller, and wherein the controller controls the gripping, moving and releasing steps of the method. This central controller may control all of the workings of the method, thereby ensuring that the method is smooth and more efficient. The controller may comprise and/or be coupled to a memory configured to store a database containing information on possible elongated medical members and/or information on previous elongated members and/or previous undertakings of the method and/or instructions for undertaking the method and/or any other suitable instructions/data. The controller may comprise and/or be coupled to a processor configured to undertake instructions stored within the memory. The central controller may be the same controller(s) as mentioned above, or have similar functioning to the controller(s) mentioned above.

In some examples, at least one of the first, second, third and fourth gripper comprises a first gripping component configured to contact at least a first portion of a respective elongated medical member on a first side of said elongated medical member; a second gripping component configured to contact at least the first portion of said elongated medical member on a second side of said elongated medical member, wherein the first side is different from the second side; a guide configured to guide, during a movement of the second gripping component, at least a first portion of the second gripping component; and an actuating component coupled to the second gripping component, wherein the second gripping component is moveable between a sixth position and a seventh position based on an actuating force provided to the second gripping component via the actuating component, and wherein at least the first portion of the second gripping component is guideable by the guide during movement of the second gripping component between the sixth position and the seventh position; wherein the first gripping component is configured to stay stationary or substantially stationary with respect to the guide, wherein the second gripping component comprises a first surface opposite to a first surface of the first gripping component, wherein, when the second gripping component is in the sixth position, said elongated medical member is grippable between the first surface of the first gripping component and the first surface of the second gripping component, and wherein, when the second gripping component is in the seventh position, said elongated medical member is not grippable between the first surface of the first gripping component and the first surface of the second gripping component. The gripper may allow for the first and/or second elongated medical members to be held in place during the process, thereby reducing the likelihood of accidental movement of the members, thereby leading to a safer method.

In some examples, the first and/or second movers comprise a linear drive unit and/or an electric motor and/or a stepper motor. The movers may be selected based on the environment of the system/apparatus undertaking the method, i.e. whether the environment is sterile, humid, hot, etc., and/or the properties of the elongated medical members. The first and/or second movers are preferably designed so that they allow for a respective gripper to move in a controlled precise manner.

In some examples, said method is undertaken manually by at least one user, and wherein the at least one user controls the gripping, moving and releasing steps of the method. This may allow for a user to finely handle the method. This may be advantageous in scenarios where the insertion is more dangerous to a patient, for example, as the user can feel haptic feedback and so, can make adjustments based on this feedback.

In some examples, at least one of the first, second, third and fourth grippers comprise a first portion of a human hand. This may be advantageous in scenarios where the insertion is more dangerous to a patient, for example, as the user can feel haptic feedback and so, can make adjustments based on this feedback.

In some examples, the first and/or second movers comprise a second portion of the human hand. This may be advantageous in scenarios where the insertion is more dangerous to a patient, for example, as the user can feel haptic feedback and so, can make adjustments based on this feedback.

In some examples, the method further comprises measuring, by the measurement sensor and/or a force sensor, a distance of insertion or a force exerted by the first elongated medical member on the second elongated medical member, respectively, and wherein upon a predetermined threshold being reached, the method is stopped. This may increase safety as the method may be stopped should the first elongated medical member be inserted too far and/or if an unexpected force is encountered and/or if the force exceeds a predetermined threshold. The force sensor may comprise a moveable member moveable between a first position and a second position. The force sensor may further comprise a resilient force member coupled to or integral to the moveable member, wherein the resilient force member is configured to provide a resilient force, when the moveable member is in the second position, to bias the moveable member towards the first position. The force sensor may further comprise a detection unit configured to detect a change in position of the moveable member from the first position to the second position and/or the second position to the first position. this may allow for the safety of the method to be improved. The method may be stopped at any time during the undertaking of the method.

In some examples, the first elongated medical member is longer than the second elongated medical member. This may allow for elongated medical members of any suitable length to use used in the method, thereby improving the flexibility and customizability of the method.

In some examples, the first elongated medical member is a guide wire and the second elongated medical member is a catheter. This may be particularly advantageous in endovascular settings. In some examples, the two elongated medical members may both be catheters. In some examples, the first elongated medical member may be a balloon catheter.

In some examples, the steps of the method are repeated until the first elongated medical member is inserted into the second elongated medical member by a predetermined distance. This may allow for the repetition of the method so that the first elongated medical member is inserted into the second elongated medical member by a sufficient distance.

In some examples, a distal tip of the second elongated medical member is kept in place during the method. This may be very important should the insertion process take place during a procedure as unintended movements of the distal tip of an elongated medical member could jeopardize the safety of a patient.

In some examples, the distal tip of the second elongated medical member is kept in place relative to a tip of a third elongated medical member during the method. This may be very important should the insertion process take place during a procedure as unintended movements of the distal tip of an elongated medical member could jeopardize the safety of a patient.

In some examples, the distal tip of the second elongated medical member is kept in place relative to a point in a blood vessel during the method. This may be very important should the insertion process take place during a procedure as unintended movements of the distal tip of an elongated medical member could jeopardize the safety of a patient.

In some examples, there may be three elongated medical members, wherein the first elongated medical member is a middle elongated medical member, the second elongated medical member is an outer elongated medical member, and a third elongated medical member is an inner elongated medical member. In this case, the first elongated medical member can be inserted into/extracted from within the second elongated medical member and the third elongated medical member can be inserted into/extracted from within the first elongated medical member. In some examples, when the first elongated medical member is being inserted/extracted, the distal tips of both the second and third elongated medical members are kept in place during the respective method. In some examples, when the third elongated medical member is being inserted/extracted, the distal tips of both the first and second elongated medical members are kept in place during the respective method.

According to a second aspect, we describe a method for extracting a first elongated medical member from within a second elongated medical member, the method comprising, gripping, by a first and a second gripper, the first and second elongated medical members at respective first and second grippers, wherein the first gripping point is a first portion of the first elongated medical member grippable by the first gripper and the second gripping point is a first portion of the second elongated medical member grippable by the second gripper; moving, by a first and second mover, the first and second grippers towards each other, wherein the first mover moves the first gripper from a first position to a second position and the second mover moves the second gripper from a third position to a fourth position; releasing the first and second grippers; gripping, by a third and a fourth gripper, the first and second elongated medical members at respective third and fourth gripping points, wherein the third gripping point is a second portion of the first elongated medical member grippable by the third gripper and the fourth gripping point is a second portion of the second elongated medical member grippable by the fourth gripper, wherein the first and second portions of the first and second elongated medical members are different respective portions; moving, by the first and second movers, the first and second grippers away from each other, wherein the first mover moves the first gripper from the second position to the first position and the second mover moves the second gripper from the fourth position to the third position; and releasing the third and fourth grippers.

The first and second elongated medical members may be any suitable members such as, for example, a catheter, a guidewire, a stent, a stent balloon, a catheter balloon or any other elongated medical member. In particular, the first and/or second elongated medical member may be a medical member suitable for endovascular procedures.

The above method may also allow for a compact apparatus that undertakes the method. For example, should the first and second elongated medical members be 120 cm in length each, a normal system would need to be at least 240 cm in length.

With this method, the overall length of the apparatus/system can be greatly reduced, thereby compacting and reducing the size of the apparatus/system using the method.

The first and second grippers may be any suitable grippers that allow for the first and second elongated medical members to be gripped at respective first and second gripping points. The first and/or second grippers may be a mechanical gripper, a magnetic gripper, or any other suitable type of gripper. In particular, the first and/or second grippers may comprise a first gripping component configured to contact at least a first portion of an elongated member on a first side of said elongated member, a second gripping component configured to contact at least the first portion of said elongated member on a second side of said elongated member, wherein the first side is different from the second side, a guide configured to guide, during a movement of the second gripping component, at least a first portion of the second gripping component and an actuating component coupled to the second gripping component, wherein the second gripping component may be moveable between a first position and a second position based on an actuating force provided to the second gripping component via the actuating component, and wherein at least the first portion of the second gripping component may be guideable by the guide during movement of the second gripping component between the first position and the second position. The first gripping component may be configured to stay stationary or substantially stationary with respect to the guide. The second gripping component may comprise a first surface opposite to a first surface of the first gripping component. When the second gripping component is in the first position, said elongated member may be grippable between the first surface of the first gripping component and the first surface of the second gripping component. When the second gripping component is in the second position, said elongated member may not grippable between the first surface of the first gripping component and the first surface of the second gripping component. The first and second grippers may allow for the first and/or second elongated medical members to be held in place during the process, thereby reducing the likelihood of accidental movement of the members, thereby leading to a safer method.

The first and second movers may allow for the first and/or second grippers to be moved. This may allow for the first and/or second elongated medical members to be gripped at different points of said members, thereby allowing for the members to be gripped at the optimal location. This may allow for a more efficient extraction of the first elongated medical member from within the second elongated medical member and for a longer lifespan of the members as the respective grippers may not impact parts of the members during the extraction.

The third and fourth grippers may be substantially the same, or the same, as the first and second grippers.

The repeating of the method may allow for the first elongated medical member to be sufficiently extracted from within the second elongated medical member, thereby allowing for the first elongated medical member to be safely stored within the second elongated medical member.

In some examples, a first pair of grippers comprises the first and third grippers, and a second pair of grippers comprises the second and fourth grippers. This may mean that the first and third grippers, and the second and fourth grippers, work as a tandem, and are dependent on each other. The actuation of each of the grippers may be controlled by a controller, with this controller also controlling the functioning of at least one of the pairs of grippers. The controller may, in some examples, be an embedded printed circuit board, PCB, controller. This controller may be placed on the same apparatus that undertakes the method. This controller may also function as a gateway/forwarder to another controller that is part of the system/apparatus, or is a controller external to the apparatus/system. A user may press a button, or other actuatable object, located on the system/apparatus which may then lead to a command to be given to the controller to start the actuation of each of the grippers. This user input can be an indication to start the insertion process according to the first aspect, an indication to start the retraction process, as described in relation to the second aspect, or any other suitable indication if a plurality of elongated medical members are inserted/extracted. The user input may, in some examples, be made from a remote location via a digital communication channel with the controller. The digital communication channel may use any known communication protocol.

In some examples, a first distance between the first and second positions, and a second distance between the third and fourth positions, are the same. This may allow for the first elongated medical member to be extracted from within the second elongated medical member in a safe manner. This may be because the movement of the first and second gripping being equal means that the effective movement of the second elongated medical member during the extraction method counteracts the movement of the first elongated medical member, thereby meaning that the first member can be extracted from within the second member in an apparatus that is compact, as mentioned above. This may also allow for a distal tip of the second elongated medical member to be kept in place during the extraction process. This may be very important should the extraction process take place during a procedure as unintended movements of the distal tip of an elongated medical member could jeopardize the safety of a patient.

In some examples, a first distance between the first and second positions, and a second distance between the third and fourth positions, are proportional to one another. This may allow for the first elongated medical member to be extracted from within the second elongated medical member in a safe manner. This may be because the movement of the first and second gripping being equal means that the effective movement of the second elongated medical member during the extraction method counteracts the movement of the first elongated medical member, thereby meaning that the first member can be extracted from within the second member in an apparatus that is compact, as mentioned above. This may be very important should the extraction process take place during a procedure as unintended movements of the distal tip of an elongated medical member could jeopardize the safety of a patient.

In some examples, the first and third grippers, and the second and fourth grippers, are comprised in respective modules. This may allow for the grippers to be protected from external factors, thereby extending their lifetimes. This may also allow for it to be easier to undertake the method as the pairs of grippers are isolated from one another, thereby reducing the likelihood of unintended physical interaction between them during the moving steps of the method. This may also allow for the pairs to be placed at different predetermined distances from each other according to the elongated medical members, thereby allowing for a customizable apparatus/system, and indeed method, as the modules and method can be used for any suitable elongated medical members. This may also allow for maintenance to be undertaken in an easier manner as the pairs of grippers are located near each other.

In some examples, the predetermined distance is determined by a measurement sensor and/or scanning a code on the first elongated medical member, wherein the code comprises information on a length of the first elongated medical member. In some examples, the distance can be calculated via an encoder, or any other suitable method, coupled to the first and/or second movers. This may mean that the system which uses the method measures the distance of extraction. Additionally or alternatively, a user of the system undertaking the method may manually enter the member length into the system and/or scan a barcode associated with the first and/or second elongated medical member. This may mean that the system can be preprogrammed to ensure the end tip of the instrument under the change procedure is never more a second predetermined distance away from the front, i.e. patient end, of the device using the method, or is always at particular place. One, or both, of these methods may be controlled by a controller. This controller may be the same controller as described above, or a separate second controller. In some examples, there may be a controller coupled to each gripper, or each pair of grippers. This controller may be responsible for giving an action to the gripper, receive input from the PCB controller mentioned above, in order to control only that gripper/pair of grippers as an independent module. In some examples, there is a module/unit controller on the apparatus/system, which is configured to control the grippers via the controller mentioned above controllers. As an example, in the present disclosure, there would be one main controller and 4 sub-controllers, where each sub-controller controls a gripper. However, there skilled person understands that there may be any number of main controllers or sub-controllers. In other words, the moving and gripping steps of the method are undertaken by at least one controller coupled to each respective gripper.

In some examples, during the method, a distal tip of the second elongated medical member is maintained in a fifth position. This may be very important should the extraction process take place during a procedure as unintended movements of the distal tip of an elongated medical member could jeopardize the safety of a patient.

In some examples, said method is undertaken by a controller, and wherein the controller controls the gripping, moving and releasing steps of the method. This central controller may control all of the workings of the method, thereby ensuring that the method is smooth and more efficient. The controller may comprise and/or be coupled to a memory configured to store a database containing information on possible elongated medical members and/or information on previous elongated members and/or previous undertakings of the method and/or instructions for undertaking the method and/or any other suitable instructions/data. The controller may comprise and/or be coupled to a processor configured to undertake instructions stored within the memory. The central controller may be the same controller(s) as mentioned above, or have similar functioning to the controller(s) mentioned above.

In some examples, at least one of the first, second, third and fourth gripper comprises a first gripping component configured to contact at least a first portion of a respective elongated medical member on a first side of said elongated medical member; a second gripping component configured to contact at least the first portion of said elongated medical member on a second side of said elongated medical member, wherein the first side is different from the second side; a guide configured to guide, during a movement of the second gripping component, at least a first portion of the second gripping component; and an actuating component coupled to the second gripping component, wherein the second gripping component is moveable between a sixth position and a seventh position based on an actuating force provided to the second gripping component via the actuating component, and wherein at least the first portion of the second gripping component is guideable by the guide during movement of the second gripping component between the sixth position and the seventh position; wherein the first gripping component is configured to stay stationary or substantially stationary with respect to the guide, wherein the second gripping component comprises a first surface opposite to a first surface of the first gripping component, wherein, when the second gripping component is in the sixth position, said elongated medical member is grippable between the first surface of the first gripping component and the first surface of the second gripping component, and wherein, when the second gripping component is in the seventh position, said elongated medical member is not grippable between the first surface of the first gripping component and the first surface of the second gripping component. The gripper may allow for the first and/or second elongated medical members to be held in place during the process, thereby reducing the likelihood of accidental movement of the members, thereby leading to a safer method.

In some examples, the first and/or second movers comprise a linear drive unit and/or an electric motor and/or a stepper motor. The movers may be selected based on the environment of the system/apparatus undertaking the method, i.e. whether the environment is sterile, humid, hot, etc., and/or the properties of the elongated medical members. The first and/or second movers are preferably designed so that they allow for a respective gripper to move in a controlled precise manner.

In some examples, said method is undertaken manually by at least one user, and wherein the at least one user controls the gripping, moving and releasing steps of the method. This may allow for a user to finely handle the method. This may be advantageous in scenarios where the extraction is more dangerous to a patient, for example, as the user can feel haptic feedback and so, can make adjustments based on this feedback.

In some examples, at least one of the first, second, third and fourth grippers comprise a first portion of a human hand. This may be advantageous in scenarios where the extraction is more dangerous to a patient, for example, as the user can feel haptic feedback and so, can make adjustments based on this feedback.

In some examples, the first and/or second movers comprise a second portion of the human hand. This may be advantageous in scenarios where the extraction is more dangerous to a patient, for example, as the user can feel haptic feedback and so, can make adjustments based on this feedback.

In some examples, the method further comprises measuring, by the measurement sensor and/or a force sensor, a distance of extraction or a force exerted by the first elongated medical member on the second elongated medical member, respectively, and wherein upon a predetermined threshold being reached, the method is stopped. This may increase safety as the method may be stopped should the first elongated medical member be extracted too far and/or if an unexpected force is encountered and/or if the force exceeds a predetermined threshold. The force sensor may comprise a moveable member moveable between a first position and a second position. The force sensor may further comprise a resilient force member coupled to or integral to the moveable member, wherein the resilient force member is configured to provide a resilient force, when the moveable member is in the second position, to bias the moveable member towards the first position. The force sensor may further comprise a detection unit configured to detect a change in position of the moveable member from the first position to the second position and/or the second position to the first position. this may allow for the safety of the method to be improved. The method may be stopped at any time during the undertaking of the method.

In some examples, the first elongated medical member is longer than the second elongated medical member. This may allow for elongated medical members of any suitable length to use used in the method, thereby improving the flexibility and customizability of the method.

In some examples, the first elongated medical member is a guide wire and the second elongated medical member is a catheter. This may be particularly advantageous in endovascular settings. The first and second elongated medical members may be any suitable members such as, for example, a catheter, a guidewire, a stent, a balloon or any other elongated medical member. In particular, the first and/or second elongated medical member may be a medical member suitable for endovascular procedures.

In some examples, the steps of the method are repeated until the first elongated medical member is extracted from the second elongated medical member by a predetermined distance. This may allow for the repetition of the method so that the first elongated medical member is extracted from within the second elongated medical member by a sufficient distance.

In some examples, a distal tip of the second elongated medical member is kept in place during the method. This may be very important should the extraction process take place during a procedure as unintended movements of the distal tip of an elongated medical member could jeopardize the safety of a patient.

In some examples, the distal tip of the second elongated medical member is kept in place relative to a tip of a third elongated medical member during the method. This may be very important should the extraction process take place during a procedure as unintended movements of the distal tip of an elongated medical member could jeopardize the safety of a patient.

In some examples, the distal tip of the second elongated medical member is kept in place relative to a point in a blood vessel during the method. This may be very important should the extraction process take place during a procedure as unintended movements of the distal tip of an elongated medical member could jeopardize the safety of a patient.

In some examples, there may be three elongated medical members, wherein the first elongated medical member is a middle elongated medical member, the second elongated medical member is an outer elongated medical member, and a third elongated medical member is an inner elongated medical member. In this case, the first elongated medical member can be inserted into/extracted from within the second elongated medical member and the third elongated medical member can be inserted into/extracted from within the first elongated medical member. In some examples, when the first elongated medical member is being inserted/extracted, the distal tips of both the second and third elongated medical members are kept in place during the respective method. In some examples, when the third elongated medical member is being inserted/extracted, the distal tips of both the first and second elongated medical members are kept in place during the respective method.

According to a third aspect, we describe a method for inserting a first elongated medical member into a second elongated medical member, the method comprising, gripping, by a first and a second gripper, the first and second elongated medical members at respective first and second gripping points, wherein the first gripping point is a first portion of the first elongated medical member grippable by the first gripper and the second gripping point is a first portion of the second elongated medical member grippable by the second gripper; moving, by a first and second mover, the first and second elongated medical members, wherein the first and second movers each comprise a pair of rollers, and wherein the first and second movers are configured to move the first and second elongated medical members via the respective pairs of rollers until the first elongated medical member is inserted into the second elongated medical member by a predetermined distance, wherein upon the insertion of the first elongated medical member into the second elongated medical member by the predetermined distance, the first and second grippers grip the first and second elongated medical members at respective third and fourth gripping points, wherein the third gripping point is a second portion of the first elongated medical member grippable by the first gripper and the fourth gripping point is a second portion of the second elongated medical member grippable by the second gripper, wherein the first and second portions of each elongated medical member are different portions.

The first and second elongated medical members may be any suitable members such as, for example, a catheter, a guidewire, a stent, a stent balloon, a catheter balloon or any other elongated medical member. In particular, the first and/or second elongated medical member may be a medical member suitable for endovascular procedures.

The above method may also allow for a compact apparatus that undertakes the method. For example, should the first and second elongated medical members be 120 cm in length each, a normal system would need to be at least 240 cm in length. With this method, the overall length of the apparatus/system can be greatly reduced, thereby compacting and reducing the size of the apparatus/system using the method.

The use of rollers may allow for a reduction in the total amount of parts used in the apparatus undertaking the method, thereby reducing the costs of the apparatus, and making maintenance of the apparatus easier. Furthermore, the modules comprising the rollers do not need to move via movers, as they move instruments by rotating the rollers. Due to this, these modules do not change their position during the procedure. This may also reduce the total amount of parts used in the apparatus undertaking the method. The rollers may be customized to the elongated medical members. That is to say, the rollers may comprise different materials, different diameters and exert different forces on the elongated medical members depending on the characteristics of the elongated medical members. The rollers may be actuated by an electric motor, a rotational drive, air/gas pressure, electromagnetic induction, or any other suitable method.

In some examples, the first gripper and the first mover are the same component, and wherein the second gripper and the second mover are the same component. That is to say, the rollers both grip the elongated medical members and move the elongated medical members. This may further reduce costs and make maintenance easier.

In some examples, said method is undertaken by a controller, and wherein the controller controls the gripping and moving steps of the method. This central controller may control all of the workings of the method, thereby ensuring that the method is smooth and more efficient. The controller may comprise and/or be coupled to a memory configured to store a database containing information on possible elongated medical members and/or information on previous elongated members and/or previous undertakings of the method and/or instructions for undertaking the method and/or any other suitable instructions/data. The controller may comprise and/or be coupled to a processor configured to undertake instructions stored within the memory. The central controller may be the same controller(s) as mentioned herein, or have similar functioning to the controller(s) mentioned herein. According to a fourth aspect, we describe a method for extracting a first elongated medical member from within a second elongated medical member, the method comprising, gripping, by a first and a second gripper, the first and second elongated medical members at respective first and second gripping points, wherein the first gripping point is a first portion of the first elongated medical member grippable by the first gripper and the second gripping point is a first portion of the second elongated medical member grippable by the second gripper; moving, by a first and second mover, the first and second elongated medical members, wherein the first and second movers each comprise a pair of rollers, and wherein the first and second movers are configured to move the first and second elongated medical members via the respective pairs of rollers until the first elongated medical member is extracted from within the second elongated medical member by a predetermined distance, wherein upon the extraction of the first elongated medical member from within the second elongated medical member by the predetermined distance, the first and second grippers grip the first and second elongated medical members at respective third and fourth gripping points, wherein the third gripping point is a second portion of the first elongated medical member grippable by the first gripper and the fourth gripping point is a second portion of the second elongated medical member grippable by the second gripper, wherein the first and second portions of each elongated medical member are different portions.

The first and second elongated medical members may be any suitable members such as, for example, a catheter, a guidewire, a stent, a stent balloon, a catheter balloon or any other elongated medical member. In particular, the first and/or second elongated medical member may be a medical member suitable for endovascular procedures.

The above method may also allow for a compact apparatus that undertakes the method. For example, should the first and second elongated medical members be 120 cm in length each, a normal system would need to be at least 240 cm in length. With this method, the overall length of the apparatus/system can be greatly reduced, thereby compacting and reducing the size of the apparatus/system using the method.

The use of rollers may allow for a reduction in the total amount of parts used in the apparatus undertaking the method, thereby reducing the costs of the apparatus, and making maintenance of the apparatus easier. Furthermore, the modules comprising the rollers do not need to move via movers, as they move instruments by rotating the rollers. Due to this, these modules do not change their position during the procedure. This may also reduce the total amount of parts used in the apparatus undertaking the method. The rollers may be customized to the elongated medical members. That is to say, the rollers may comprise different materials, different diameters and exert different forces on the elongated medical members depending on the characteristics of the elongated medical members. The rollers may be actuated by an electric motor, a rotational drive, air/gas pressure, electromagnetic induction, or any other suitable method.

In some examples, the first gripper and the first mover are the same component, and wherein the second gripper and the second mover are the same component. That is to say, the rollers both grip the elongated medical members and move the elongated medical members. This may further reduce costs and make maintenance easier.

In some examples, said method is undertaken by a controller, and wherein the controller controls the gripping and moving steps of the method. This central controller may control all of the workings of the method, thereby ensuring that the method is smooth and more efficient. The controller may comprise and/or be coupled to a memory configured to store a database containing information on possible elongated medical members and/or information on previous elongated members and/or previous undertakings of the method and/or instructions for undertaking the method and/or any other suitable instructions/data. The controller may comprise and/or be coupled to a processor configured to undertake instructions stored within the memory. The central controller may be the same controller(s) as mentioned herein, or have similar functioning to the controller(s) mentioned herein.

Although rollers are mentioned with respect to at least the third and fourth aspects, the rollers may be not only wheels, but any other movement solution which allows the first and/or second elongated medical members to move linearly, but wherein the module that comprises the movement solution stays in place. That is to say, a rotating transportation belt, a pinion, or any other suitable solution may be used.

According to a fifth aspect, we describe a method for inserting a first elongated medical member into a second elongated medical member and extracting the first elongated medical member from within the second elongated medical member, the method comprising: the method according to the first aspect or the third aspect; and the method according to the second aspect or the fourth aspect.

According to a sixth aspect, we describe a medical apparatus for inserting a first elongated medical member into a second elongated medical member and/or extracting the first elongated medical member from within the second elongated medical member, the apparatus comprising: a first module and a second module, wherein each of the first and second module comprises a corresponding, respective opening for the first elongated medical member to be passed through the first and second modules, and wherein the second elongated medical member is passed through the opening of at least the second module; a first movement unit configured to move at least a portion of the first module in a first direction and in a second direction, wherein the first direction is opposite to the second direction; a second movement unit configured to move at least a portion of the second module in the first direction and in the second direction; at least four gripping units, wherein the first module comprises at least a first and a second gripping unit, and wherein the second module comprises at least a third gripping unit and a fourth gripping unit, wherein the first and second gripping units are configured to grip a first and a second portion of the first elongated medical member, respectively, and wherein the third and fourth gripping units are configured to grip a first and a second portion of the second elongated medical member, respectively, wherein a) the first and second elongated medical members are configured to be gripped at respective first and second gripping points by the first and third gripping units, respectively; wherein the first movement unit is configured to move the first gripping unit in the first direction from a first position to a second position, and wherein the second movement unit is configured to move the third gripping unit in the second direction from a third position to a fourth position; wherein the first and third gripping units are configured to release the first and second elongated medical members, respectively; and the first and second elongated medical members are configured to be gripped at respective third and fourth gripping points by the second and fourth gripping units; wherein the first movement unit is configured to move the first gripping unit in the second direction from the second position to the first position, and wherein the second movement unit is configured to move the third gripping unit in the first direction from the fourth position to the third position and/or b) the first and second elongated medical members are configured to be gripped at respective first and second gripping points by the first and third gripping units, respectively; wherein the first movement unit is configured to move the first gripping unit in the second direction from the second position to the first position, and wherein the second movement unit is configured to move the third gripping unit in the first direction from the fourth position to the third position; wherein the first and third gripping units are configured to release the first and second elongated medical members, respectively; and the first and second elongated medical members are configured to be gripped at respective third and fourth gripping points by the second and fourth gripping units; wherein the first movement unit is configured to move the first gripping unit in the first direction from the first position to the second position, and wherein the second movement unit is configured to move the third gripping unit in the second direction from the third position to the fourth position.

In some examples, the first and/or the second module comprises a first gripper replacing the gripping units in the respective module and a first mover replacing the movement unit configured to move at least the portion of the respective module, wherein the first and/or second module comprises: a first gripper configured to grip the respective elongated medical member at a respective first gripping point, wherein the first gripping point is a first portion of the respective elongated medical member grippable by the first gripper; a first mover configured to move the respective elongated medical member, wherein the first mover comprises a pair of rollers, and wherein the first mover is configured to move the respective elongated medical member via the pair of rollers, wherein, upon the insertion of the first elongated medical member into the second elongated medical member by the predetermined distance or the extraction of the first elongated medical member from within the second elongated medical member by the predetermined distance, the first gripper is further configured to grip the respective elongated medical member at a respective second gripping point, wherein the second gripping point is a second portion of the respective elongated medical member grippable by the first gripper, wherein the first and second portions of each elongated medical member are different portions.

The use of rollers may allow for a reduction in the total amount of parts used in the apparatus undertaking the method, thereby reducing the costs of the apparatus, and making maintenance of the apparatus easier. Furthermore, the modules comprising the rollers do not need to move via movers, as they move instruments by rotating the rollers. Due to this, these modules do not change their position during the procedure. This may also reduce the total amount of parts used in the apparatus undertaking the method.

According to an additional aspect, we describe a medical apparatus for inserting a first elongated medical member into a second elongated medical member and/or extracting the first elongated medical member from within the second elongated medical member, wherein the apparatus is configured to perform the method of any one of the first to fifth aspects.

In some examples, a length of the medical apparatus is less than a total length of the first and second elongated medical members, or is less than a length of the longest one of the first and second elongated medical members. This may allow for a compact apparatus. For example, should the first and second elongated medical members be 120 cm in length each, a normal system would need to be at least 240 cm in length. With this apparatus, the overall length of the apparatus can be greatly reduced, thereby compacting and reducing the size of the apparatus.

The units mentioned above are suitable to undertake the respective parts of the method of any one of the first to fourth aspects described above.

In some examples, the aspects described herein can take place outside of the human body and/or animal body. That is to say, the aspects as described herein may be used on and/or in an artificial structure for the purpose of training surgeons, doctors and other medical staff. The artificial structure may resemble at least a part of an (endo)vascular system for the purposes of said training.

The aspects described herein may function by inserting/extracting the first elongated medical member into/from the second elongated medical member via inducing slack in the second elongated medical member. That is, the first elongated medical member may be inserted into the second member by essentially pulling the second elongated medical member over the first elongated medical member, thereby allowing for the first elongated medical member to be inserted into the second elongated medical member. The reverse may happen for the extraction process.

In a further example of the functioning of the aspects as described herein, particularly in relation to the third and fourth aspects, the rollers and/or the first and/or second grippers may grip different parts of the first and/or second elongated medical members during the insertion/extraction processes. The rollers and/or the first and/or second grippers may grip different parts of the members at the beginning and end of the processes. In other words, as the second elongated medical member is pulled over the first elongated medical member, the roller and/or gripper for the second elongated medical member may grip different parts of the second elongated medical member at the beginning and end of the process. Additionally, as the distal end of the first elongated medical member may be kept in place during the insertion process, the rollers and/or the first and/or second grippers for the first elongated medical member may move the first elongated medical member in order to counteract the movement of the second elongated medical member and/or the rollers and/or the first and/or second grippers related to the second elongated medical member and so, the roller and/or gripper for the first elongated medical member may grip different parts of the first elongated medical member at the beginning and end of the process.

Any advantages and features described in relation to the any of the above aspects and examples may be realized in any of the other aspects and examples described above. That is to say, for example, any aspects described with respect to the first or second aspects can be introduced into the third and fourth aspects should they be compatible with the third and fourth aspects.

It is clear to a person skilled in the art that certain features of the system set forth herein may be implemented under use of hardware (circuits), software means, or a combination thereof. The software means can be related to programmed microprocessors or a general computer, an ASIC (Application Specific Integrated Circuit) and/or DSPs (Digital Signal Processors). For example, a processing unit may be implemented at least partially as a computer, a logical circuit, an FPGA (Field Programmable Gate Array), a processor (for example, a microprocessor, microcontroller (μC) or an array processor)/a core/a CPU (Central Processing Unit), an FPU (Floating Point Unit), NPU (Numeric Processing Unit), an ALU (Arithmetic Logical Unit), a Coprocessor (further microprocessor for supporting a main processor (CPU)), a GPGPU (General Purpose Computation on Graphics Processing Unit), a multi-core processor (for parallel computing, such as simultaneously performing arithmetic operations on multiple main processor(s) and/or graphical processor(s)) or a DSP.

Even if some of the aspects described above have been described in reference to any one of the first to fifth aspects, these aspects may also apply to an apparatus/system (in particular an apparatus for endovascular procedures) and vice versa.

Furthermore, although the terms “for inserting a first elongated medical member into a second elongated medical member” and “for extracting a first elongated medical member from within a second elongated medical member” are used, it is to be understood that the above aspects may also apply to a method, and device, for placing a first elongated medical member over a second elongated medical member, and removing a first elongated medical member from over a second elongated medical member. In this case, the first and second elongated medical members may be at least partially adjacent to one another.

Throughout the disclosure, the terms “instrument” and “elongated member”, and the terms “movement unit” and “mover” may mean the same thing and may be interchangeable.

### BRIEF DESCRIPTION OF THE DRAWINGS

These and other aspects of the invention will now be further described, by way of example only, with reference to the accompanying figures, wherein like reference numerals refer to like parts, and in which:

FIG. 1 shows a schematic view of an apparatus for inserting a first elongated medical member into a second elongated medical member according to some example implementations as described herein;

FIG. 2 shows a schematic view of an apparatus for extracting a first elongated medical member from within a second elongated medical member according to some example implementations as described herein;

FIG. 3 shows a schematic view of an apparatus for inserting a first elongated medical member into a second elongated medical member according to some example implementations as described herein;

FIG. 4 shows a schematic view of an apparatus for extracting a first elongated medical member from within a second elongated medical member according to some example implementations as described herein;

FIG. 5 shows flow chart of a method for inserting a first elongated medical member into a second elongated medical member according to some example implementations as described herein;

FIG. 6 shows flow chart of a method for extracting a first elongated medical member from within a second elongated medical member according to some example implementations as described herein;

FIG. 7 shows flow chart of a method for inserting a first elongated medical member into a second elongated medical member according to some example implementations as described herein;

FIG. 8 shows flow chart of a method for extracting a first elongated medical member from within a second elongated medical member according to some example implementations as described herein.

### DETAILED DESCRIPTION OF THE PREFERRED EMBODIMENTS

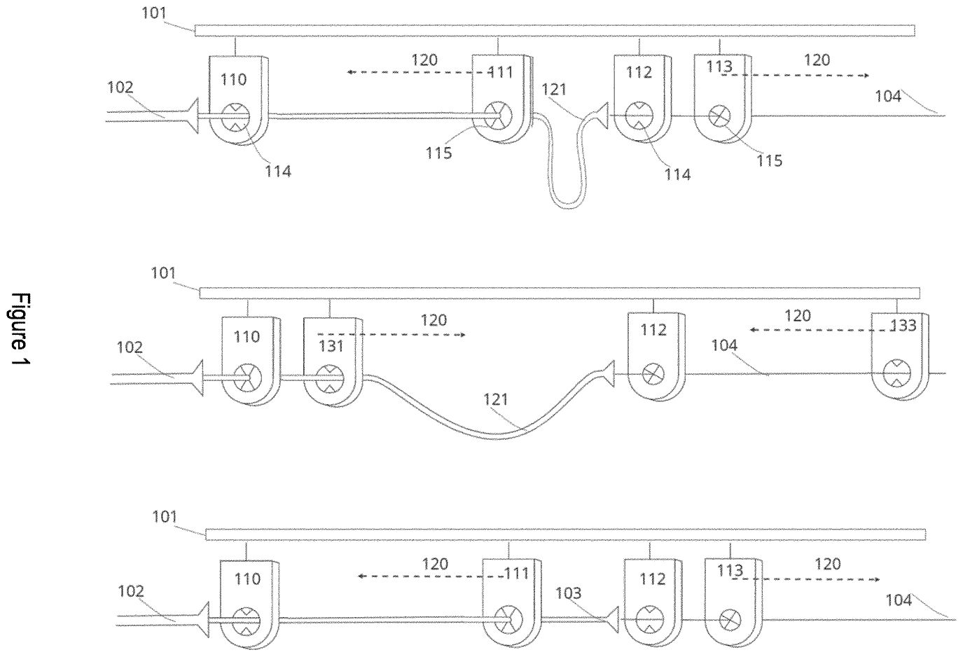

FIG. 1 shows a schematic view of an apparatus for inserting a first elongated medical member into a second elongated medical member according to some example implementations as described herein.

The apparatus for insertion of a first elongated medical member 103 into a second elongated medical member 102, along with a third elongated medical member 104, is shown in FIG. 1. The apparatus comprises a base 101 with, in this case, a linear drive, but the skilled person understands that any sort of drive is suitable for the apparatus. The apparatus further comprises four gripping units 110, 111, 112, 113, but the skilled person understands that there may be more, or fewer, gripping units should they be needed, or if the insertion is happening to multiple elongated medical members at the same time.

In the first step, the second and fourth gripping units 111, 113 are configured to grip 115 the respective elongated medical members 103, 105 at first and second gripping points. The first and third gripping units 110, 112 are configured to not grip 114 the elongated medical members 103, 104.

The second and fourth gripping units 111, 113 are then moved, via the base 101 comprising the linear drive, in the direction of the arrows 120 from respective first and second positions to respective third and fourth positions while still gripping their respective elongated medical members 103, 104. It is then seen that there is less slack 121 in the first elongated medical member 103. In a particular example, the second and fourth gripping units 111, 113 may be moved the same distance.

In the second step, the second and fourth gripping units 111, 113 release their respective elongated medical members 103, 104 and the first and third gripping units 110, 112 grip the elongated medical members 103, 104 which the second and fourth gripping units 111, 113 released. In some examples, the gripping and releasing may take place simultaneously, in sequential order, or the other way around from what is described here and in sequential order.

While the elongated medical members are gripped by the first and third gripping units 110, 112, the second and fourth gripping units move from their respective third and fourth positions 131, 133 back to their respective first and second positions 111, 113 in the direction of the arrows 120.

The first and third gripping units 110, 112 then release the respective elongated medical members and the process repeats until there is no, or little, or a predetermined amount of, slack 121 in the first elongated medical member 103, thereby indicating that the first elongated medical member 103 has been fully inserted.

The above process may allow for a distal tip of at least one of the other elongated medical members 102, 104 to stay in place during the insertion process. This may be very important should the extraction process take place during a procedure as unintended movements of the distal tip of an elongated medical member could jeopardize the safety of a patient.

In some examples, the second and/or fourth gripping units 111, 113 may comprise a measurement sensor and/or a force sensor (not shown) configured to measure a distance of insertion or a force exerted by the first elongated medical member on the second elongated medical member, respectively, and wherein upon a predetermined threshold being reached, the process is stopped. This may increase safety as the process may be stopped should the first elongated medical member 103 be inserted too far and/or if an unexpected force is encountered and/or if the force exceeds a predetermined threshold. The force sensor may comprise a moveable member moveable between a first position and a second position. The force sensor may further comprise a resilient force member coupled to or integral to the moveable member, wherein the resilient force member is configured to provide a resilient force, when the moveable member is in the second position, to bias the moveable member towards the first position. The force sensor may further comprise a detection unit configured to detect a change in position of the moveable member from the first position to the second position and/or the second position to the first position. this may allow for the safety of the method to be improved.

In the above process, the first elongated medical member 103 is inserted a predetermined distance into at least one of the other elongated medical members 102, 104. This distance may be determined by a same or different measurement sensor as the one described above and/or scanning a code on the first elongated medical member (not shown), wherein the code comprises information on a length of the first elongated medical member. In some examples, the distance can be calculated via an encoder, or any other suitable method, coupled to the base 101 and/or at least one of the second and fourth gripping units 111, 113. This may mean that the system apparatus measures the distance of insertion. Additionally or alternatively, a user of the apparatus may manually enter the member 102, 103, 104 length into the a controller of the apparatus (not shown) and/or scan a barcode associated with at least one of the elongated medical members 102, 103, 104. This may mean that the apparatus can be preprogrammed to ensure the distal tip of one of the other elongated medical members 102, 104 is never more a second predetermined distance away from the front, i.e. patient end, of the apparatus, or is always at particular place. One, or both, of these methods may be controlled by a controller.

The “freezing” in place of the distal tip of one of the other elongated medical members 102, 104, as described above, even occurs when the length of the elongated medical members 102, 103, 104 is longer than available distance. That is to say, even when there is slack 121 in the first elongated medical member 103, the distal tip of one of the other elongated medical members 102, 104 remains in place. The above functionality is ensured by the four gripping units being part of two modules, wherein a first module comprises the first and second gripping units 110, 111 and the second module comprises the third and fourth gripping units 112, 113. Inserting the first elongated medical member 103 allows for the distal tip of one of the other elongated medical members 102, 104 to remain in place via the second and fourth gripping units 111, 113 moving the same distance, and at the same speed, when gripping their respective elongated members 103, 104. This is further ensured by the movement of the second and fourth gripping units 111, 113 in opposite directions, as can be seen by the arrows 120 of FIG. 1.

The elongated medical members 102, 103, 104 may comprise, for example, a guide wire, a stent, a catheter, a balloon or any other suitable instrument. The process described above may be used to, for example, change any of these elongated medical members for a second elongated medical member of the same type, or of a different type.

As the movement of the second and fourth gripping units 111, 113 is limited, the above process may be carried out a theoretical unlimited amount of times until the first elongated medical member 103 is adequately inserted.

This principle allows to have all three, in the present example, elongated medical members 102, 103, 104 looped/buckled between the modules so that the overall apparatus length is short, despite the long length of the elongated medical members 102, 103, 104. Indeed, the length of at least one of the elongated medical members 102, 103, 104 may be longer than the length of the apparatus.

The above process also allows for a “freezing” dynamic between elongated medical members 102, 103, 104. In other words, the above movement allows for the distal tip of one of the other elongated medical members 102, 104 to remain in the same position relative to a point in a vessel, should one of the other elongated medical members 102, 104 be within a patient at the time of the process. This same principle applies any of the elongated medical members 102, 103, 104 that are not the first elongated medical member 103.

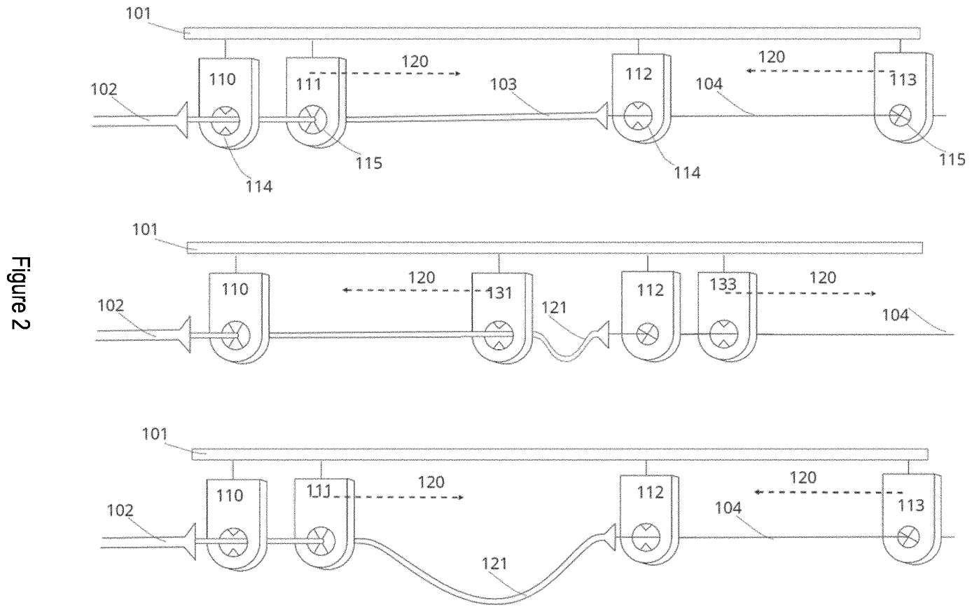

FIG. 2 shows a schematic view of an apparatus for extracting a first elongated medical member from within a second elongated medical member according to some example implementations as described herein.

The apparatus for retraction of a first elongated medical member 103 from within a second elongated medical member 102, along with a third elongated medical member 104, is shown in FIG. 2. The apparatus comprises a base 101 with, in this case, a linear drive, but the skilled person understands that any sort of drive is suitable for the apparatus. The apparatus further comprises four gripping units 110, 111, 112, 113, but the skilled person understands that there may be more gripping units should they be needed, or if the extraction is happening to multiple elongated medical members at the same time.

In the first step, the second and fourth gripping units 111, 113 are configured to grip 115 the respective elongated medical members 103, 105 at first and second gripping points. The first and third gripping units 110, 112 are configured to not grip 114 the elongated medical members 103, 104.

The second and fourth gripping units 111, 113 are then moved, via the base 101, in the direction of the arrows 120 from respective first and second positions to respective third and fourth positions while still gripping their respective elongated medical members 103, 104. It is then seen that there is less slack 121 in the first elongated medical member 103. In a particular example, the second and fourth gripping units 111, 113 may be moved the same distance.

In the second step, the second and fourth gripping units 111, 113 release their respective elongated medical members 103, 104 and the first and third gripping units 110, 112 grip the elongated medical members 103, 104 which the second and fourth gripping units 111, 113 released. In some examples, the gripping and releasing may take place simultaneously, in sequential order, or the other way around in sequential order.

While the elongated medical members are gripped by the first and third gripping units 110, 112, the second and fourth gripping units move from their respective third and fourth positions 131, 133 back to their respective first and second positions 111, 113 in the direction of the arrows 120.

The first and third gripping units 110, 112 then release the respective elongated medical members and the process repeats until there is a predetermined amount of slack 121 in the first elongated medical member 103, thereby indicating that the first elongated medical member 103 has been fully extracted. This then allows for the first elongated medical member 103 to be manually extracted from the apparatus, if needed.

It can therefore be seen that the process of FIG. 2 is similar to that of FIG. 1, but the movement of the second and fourth gripping units 111, 113 when they are gripping their respective elongated medical members 103, 104 in FIG. 2 is in the opposite direction of that of FIG. 1.

Therefore, the apparatus of FIG. 2 is substantially similar, if not the same, as the apparatus of FIG. 1 and so, may comprise the same features and have the same effects as the apparatus of FIG. 1.

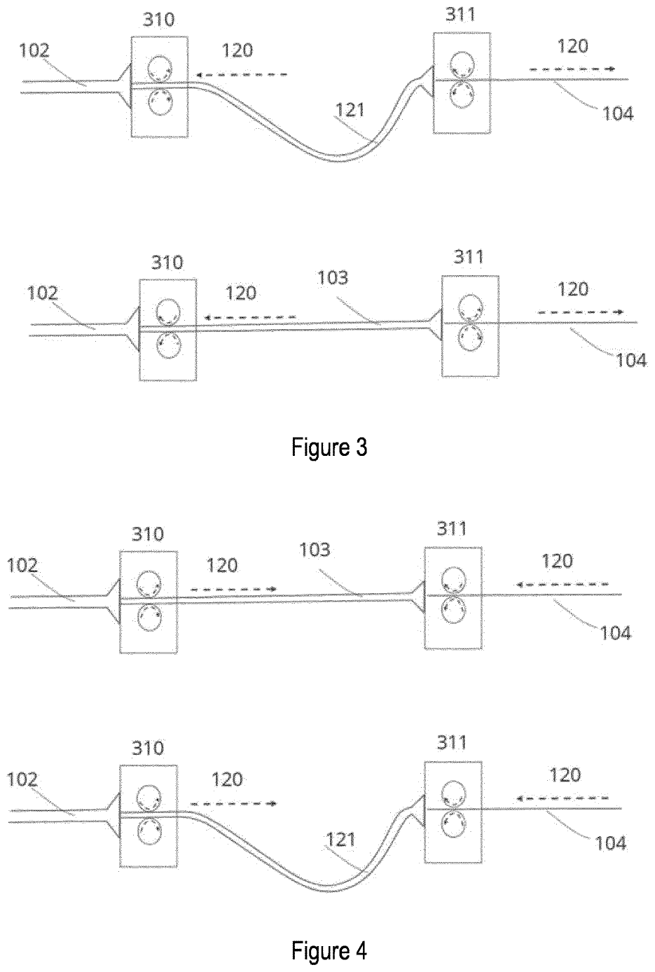

FIG. 3 shows a schematic view of an apparatus for inserting a first elongated medical member into a second elongated medical member according to some example implementations as described herein.

FIG. 3 shows a second method of inserting a first elongated medical member 103 into a second elongated medical member 102, along with a third elongated medical member 104.

In this example, there are two modules 310, 311, wherein the modules are each configured to grip a respective one of the elongated medical members 103, 104.

In a first step of the process, the rollers within the modules 310, 311 rotate, via a drive, so that the elongated medical members 103, 104 which the modules grip 310, 311 move in the direction shown by the arrows 120. This continues until there is no, or little, or a predetermined, amount of slack 121 in the first elongated medical member 103.

It can therefore be seen that in FIG. 3, the same essential process as in FIG. 1 is undertaken, but with rollers instead of gripping units 110, 111, 112, 113. Therefore, the modules may have all, or at least some, of the same features as mentioned in relation to FIG. 1, and have all, or at least some, of the same technical effects as the process and apparatus described in FIG. 1.

Furthermore, in the example of FIG. 3, the gripping and moving units are the same. That is to say the modules 310, 311 both grip their respective elongated medical members 102, 103, 104 and move said elongated medical members 102, 103, 104. Therefore, the modules 310, 311, and the rollers within them, act as both the grippers and the movers.

FIG. 4 shows a schematic view of an apparatus for extracting a first elongated medical member from within a second elongated medical member according to some example implementations as described herein.

FIG. 4 shows a second method of extracting a first elongated medical member 103 from within a second elongated medical member 102, along with a third elongated medical member 104.

In this example, there are two modules 310, 311, wherein the modules are each configured to grip a respective one of the elongated medical members 103, 104.

In a first step of the process, the rollers within the modules 310, 311 rotate, via a drive, so that the elongated medical members 103, 104 which the modules grip 310, 311 move in the direction shown by the arrows 120. This continues until there is a predetermined amount of slack 121 in the first elongated medical member 103. This then allows for the first elongated medical member 103 to be manually extracted from the apparatus, if needed.

Furthermore, in the example of FIG. 4, the gripping and moving units are the same. That is to say the modules 310, 311 both grip their respective elongated medical members 102, 103, 104 and move said elongated medical members 102, 103, 104.

Therefore, the modules 310, 311, and the rollers within them, act as both the grippers and the movers.

It can therefore be seen that the process of FIG. 4 is similar to that of FIG. 3, but the movement of the rollers being in the opposite direction of that of FIG. 3.

Therefore, the apparatus of FIG. 4 is substantially similar, if not the same, as the apparatus of FIG. 3 and so, may comprise the same features and have the same effects as the apparatus of FIG. 3.

In some examples of the apparatus of FIG. 3 or 4, the modules 310, 311 themselves may be able to move via a base with a linear drive, or any other suitable type of drive, like that shown in FIGS. 1 and 2.

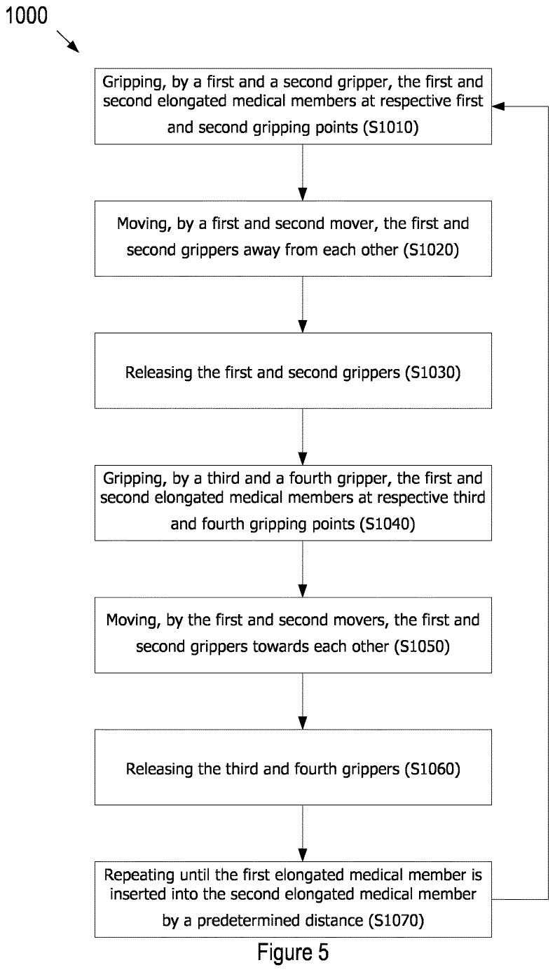

FIG. 5 shows flow chart of a method for inserting a first elongated medical member into a second elongated medical member according to some example implementations as described herein.

The flow chart of FIG. 5 shows the process of the apparatus in FIG. 1.

The method 1000 comprises seven main steps:
1) The first and second elongated medical members are gripped by first and second grippers at respective first and second gripping points S1010;
2) The first and second grippers are then moved by first and second movers away from each other S1020;
3) The first and second grippers are then released S1030;
4) The first and second elongated medical members are then gripped by a third and a fourth gripper at respective third and fourth gripping points S1040;
5) The first and second grippers are then moved by the first and second movers towards each other S1050;
6) The third and fourth grippers are then released S1060; and
7) This is repeated until the first elongated medical member is inserted into the second elongated medical member by a predetermined distance S1070.

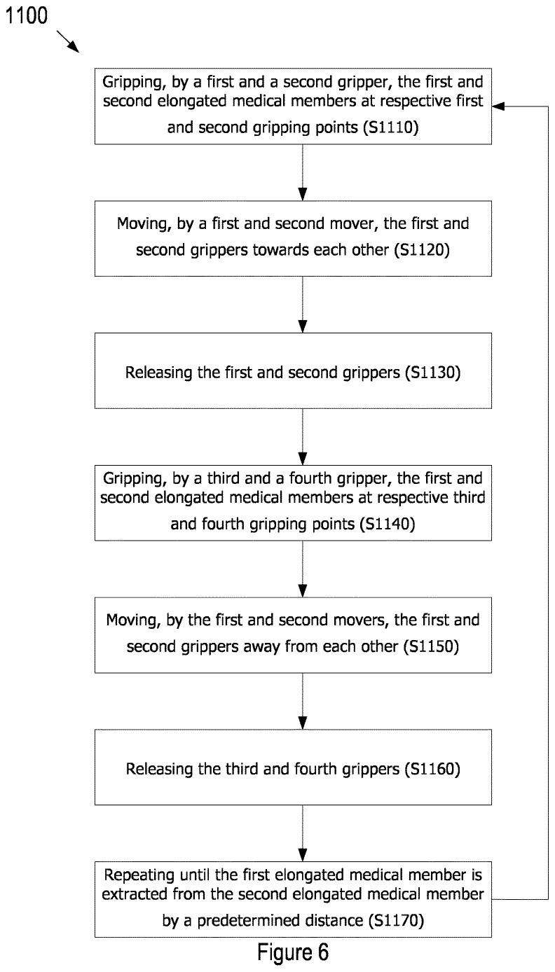

FIG. 6 shows flow chart of a method for extracting a first elongated medical member from within a second elongated medical member according to some example implementations as described herein.

The flow chart of FIG. 6 shows the process of the apparatus in FIG. 2.

The method 1100 comprises seven main steps:
1) The first and second elongated medical members are gripped by first and second grippers at respective first and second gripping points S1110;
2) The first and second grippers are then moved by first and second movers towards each other S1120;
3) The first and second grippers are then released S1130;
4) The first and second elongated medical members are then gripped by a third and a fourth gripper at respective third and fourth gripping points S1140;
5) The first and second grippers are then moved by the first and second movers away from each other S1150;
6) The third and fourth grippers are then released S1160; and
7) This is repeated until the first elongated medical member is inserted into the second elongated medical member by a predetermined distance S1170.

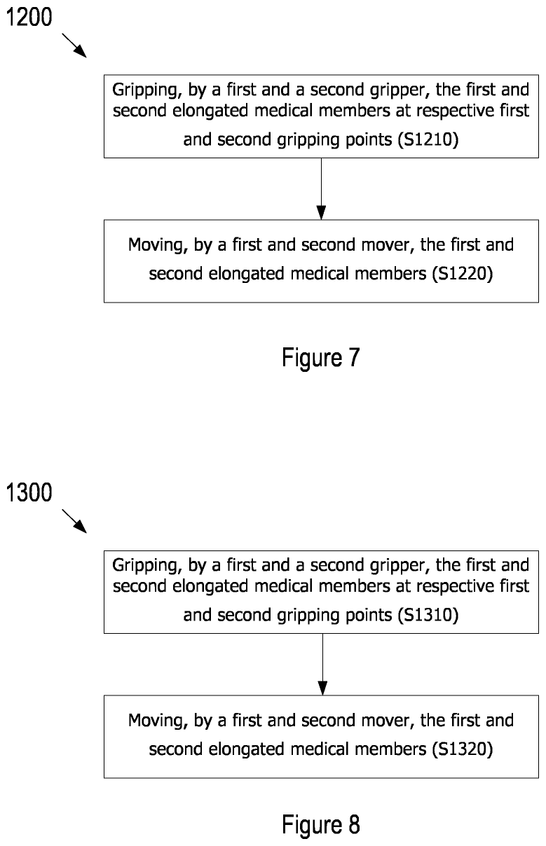

FIG. 7 shows flow chart of a method for inserting a first elongated medical member into a second elongated medical member according to some example implementations as described herein.

The flow chart of FIG. 7 shows the process of the apparatus in FIG. 3.

The method 1200 comprises two main steps:
1) The first and second elongated medical members are gripped by first and second grippers at respective first and second gripping points S1210; and
2) The first and second elongated medical members are moved by first and second movers S1220.

FIG. 8 shows flow chart of a method for extracting a first elongated medical member from within a second elongated medical member according to some example implementations as described herein.

The flow chart of FIG. 8 shows the process of the apparatus in FIG. 4.

The method 1300 comprises two main steps:
1) The first and second elongated medical members are gripped by first and second grippers at respective first and second gripping points S1310; and
2) The first and second elongated medical members are moved by first and second movers S1320.

In addition to the above examples, in some examples, there may be both roller modules 310, 311, as described in FIGS. 3 and 4, as well as gripping units 110, 111, 112, 113 as described in relation to FIGS. 1 and 2. That is to say one of the roller modules 310 311 may be replaced by the first and second gripping units 110, 111 or the third and fourth gripping units 112, 113, wherein each part maintains the same functionality as described in their respective examples. This may allow for a more flexible and customizable apparatus and allow for improved safety, reduced costs, and a longer lifetime of the elongated medical members 102, 103, 104.

In some examples, the apparatuses described herein are robotic systems/apparatuses/devices. This may allow for the apparatus, processes and methods to be undertaken from a remote location. This may allow for improved patient outcomes as the user of the apparatus does not need to be in the same location as the patient and so, the user can work on patients in several location simultaneously, or perform an emergency process on a patient in a location where there is not a qualified user.

In some examples, an apparatus may comprise both the gripping units variant as described in FIGS. 1 and 2, and the roller module variant as described in FIGS. 3 and 4. In some examples, the apparatus may switch between the two variants during the insertion/extraction of an elongated medical member 103. For example, the majority of the process may be undertaken by the gripping units variant, and then the roller module variant may be used for fine tuning the insertion/extraction by providing micro movements, or vice versa. In some examples, the insertion may be undertaken by the gripping units variant and the extraction by the roller module variant, or vice versa.

Although only two modules are described in the above examples, the killed person understands that there may be any number of modules according to the number of elongated medical members 102, 103, 104 being inserted/extracted, and the type of elongated medical members 102, 103, 104 being used in the methods/processes.

1. A medical apparatus for inserting a first elongated medical member into a second elongated medical member and/or extracting the first elongated medical member from within the second elongated medical member, the apparatus comprising:
a first module and a second module, wherein each of the first and second module comprises a corresponding, respective opening for the first elongated medical member to be passed through the first and second modules, and wherein the second elongated medical member is passed through the opening of at least the second module;
a first movement unit configured to move at least a portion of the first module in a first direction and in a second direction, wherein the first direction is opposite to the second direction;
a second movement unit configured to move at least a portion of the second module in the first direction and in the second direction;
at least four gripping units, wherein the first module comprises at least a first and a second gripping unit, and wherein the second module comprises at least a third gripping unit and a fourth gripping unit, wherein the first and second gripping units are configured to grip a first and a second portion of the first elongated medical member, respectively, and wherein the third and fourth gripping units are configured to grip a first and a second portion of the second elongated medical member, respectively,
wherein
a) the first and second elongated medical members are configured to be gripped at respective first and second gripping points by the first and third gripping units, respectively;
wherein the first movement unit is configured to move the first gripping unit in the first direction from a first position to a second position, and wherein the second movement unit is configured to move the third gripping unit in the second direction from a third position to a fourth position;
wherein the first and third gripping units are configured to release the first and second elongated medical members, respectively; and
the first and second elongated medical members are configured to be gripped at respective third and fourth gripping points by the second and fourth gripping units;
wherein the first movement unit is configured to move the first gripping unit in the second direction from the second position to the first position, and wherein the second movement unit is configured to move the third gripping unit in the first direction from the fourth position to the third position
and/or
b) the first and second elongated medical members are configured to be gripped at respective first and second gripping points by the first and third gripping units, respectively;
wherein the first movement unit is configured to move the first gripping unit in the second direction from the second position to the first position, and wherein the second movement unit is configured to move the third gripping unit in the first direction from the fourth position to the third position;
wherein the first and third gripping units are configured to release the first and second elongated medical members, respectively; and
the first and second elongated medical members are configured to be gripped at respective third and fourth gripping points by the second and fourth gripping units;
wherein the first movement unit is configured to move the first gripping unit in the first direction from the first position to the second position, and wherein the second movement unit is configured to move the third gripping unit in the second direction from the third position to the fourth position.

2. The medical apparatus according to claim 1, wherein the first and/or the second module comprises a first gripper replacing the gripping units in the respective module and a first mover replacing the movement unit configured to move at least the portion of the respective module, wherein the first and/or second module comprises:
a first gripper configured to grip the respective elongated medical member at a respective first gripping point, wherein the first gripping point is a first portion of the respective elongated medical member grippable by the first gripper;
a first mover configured to move the respective elongated medical member, wherein the first mover comprises a pair of rollers, and wherein the first mover is configured to move the respective elongated medical member via the pair of rollers,
wherein, upon the insertion of the first elongated medical member into the second elongated medical member by the predetermined distance or the extraction of the first elongated medical member from within the second elongated medical member by the predetermined distance, the first gripper is further configured to grip the respective elongated medical member at a respective second gripping point, wherein the second gripping point is a second portion of the respective elongated medical member grippable by the first gripper, wherein the first and second portions of each elongated medical member are different portions.

3. The medical apparatus according to claim 1, wherein a length of the medical apparatus is less than a total length of the first and second elongated medical members, or is less than a length of the longest one of the first and second elongated medical members.

4. A method for inserting a first elongated medical member into a second elongated medical member or extracting the first elongated medical member from within the second elongated medical member, the method comprising:
gripping, by a first and a second gripper, the first and second elongated medical members at respective first and second gripping points, wherein the first gripping point is a first portion of the first elongated medical member grippable by the first gripper and the second gripping point is a first portion of the second elongated medical member grippable by the second gripper;
moving, by a first and second mover, the first and second grippers away from or towards each other, wherein the first mover moves the first gripper from a first position to a second position and the second mover moves the second gripper from a third position to a fourth position;
releasing the first and second grippers;
gripping, by a third and a fourth gripper, the first and second elongated medical members at respective third and fourth gripping points, wherein the third gripping point is a second portion of the first elongated medical member grippable by the third gripper and the fourth gripping point is a second portion of the second elongated medical member grippable by the fourth gripper, wherein the first and second portions of the first and second elongated medical members are different respective portions;
moving, by the first and second movers, the first and second grippers towards or away from each other, wherein the first mover moves the first gripper from the second position to the first position and the second mover moves the second gripper from the fourth position to the third position; and
releasing the third and fourth grippers.

5. The method of claim 4, wherein a first pair of grippers comprises the first and third grippers, and a second pair of grippers comprises the second and fourth grippers.

6. The method of claim 4, wherein a first distance between the first and second positions, and a second distance between the third and fourth positions, are the same and/or are proportional to one another.

7. (canceled)

8. The method of claim 4, wherein the first and third grippers, and the second and fourth grippers, are comprised in respective modules;
and/or
wherein the predetermined distance is determined by a measurement sensor and/or scanning a code on the first elongated medical member, wherein the code comprises information on a length of the first elongated medical member;
and/or
wherein, during the method, a distal tip of the second elongated medical member is maintained in a fifth position.

9-10. (canceled)

11. The method of claim 4, wherein said method is undertaken by a controller, and wherein the controller controls the gripping, moving and releasing steps of the method.

12. The method of claim 11, wherein at least one of the first, second, third and fourth grippers comprises a first gripping component configured to contact at least a first portion of a respective elongated medical member on a first side of said elongated medical member;
a second gripping component configured to contact at least the first portion of said elongated medical member on a second side of said elongated medical member, wherein the first side is different from the second side;
a guide configured to guide, during a movement of the second gripping component, at least a first portion of the second gripping component; and
an actuating component coupled to the second gripping component, wherein the second gripping component is moveable between a sixth position and a seventh position based on an actuating force provided to the second gripping component via the actuating component, and wherein at least the first portion of the second gripping component is guideable by the guide during movement of the second gripping component between the sixth position and the seventh position;
wherein the first gripping component is configured to stay stationary or substantially stationary with respect to the guide,
wherein the second gripping component comprises a first surface opposite to a first surface of the first gripping component,
wherein, when the second gripping component is in the sixth position, said elongated medical member is grippable between the first surface of the first gripping component and the first surface of the second gripping component, and
wherein, when the second gripping component is in the seventh position, said elongated medical member is not grippable between the first surface of the first gripping component and the first surface of the second gripping component.

13. (canceled)

14. The method of claim 4, wherein said method is undertaken manually by at least one user, and wherein the at least one user controls and/or performs one or more of the gripping, moving and releasing steps of the method.

15. The method of claim 4, the method further comprises measuring, by the measurement sensor and/or a force sensor, a distance of insertion or a force exerted by the first elongated medical member on the second elongated medical member, respectively, and wherein, upon a predetermined threshold being reached, the method is stopped.

16. The method of claim 4, wherein the first elongated medical member is longer than the second elongated medical member;
and/or
wherein the first elongated medical member is a guide wire and the second elongated medical member is a catheter;
and/or
wherein the steps of the method are repeated until the first elongated medical member is inserted into or extracted from the second elongated medical member by a predetermined distance.

17-18. (canceled)

19. The method of claim 4, wherein a distal tip of the second elongated medical member is kept in place during the method.

20. The method of claim 19, wherein the distal tip of the second elongated medical member is kept in place relative to a tip of a third elongated medical member during the method, and/or wherein the distal tip of the second elongated medical member is kept in place relative to a point in a blood vessel during the method.

21-39. (canceled)

40. A method for inserting a first elongated medical member into a second elongated medical member or for extracting the first elongated medical member from within a second elongated medical member, the method comprising:
gripping, by a first and a second gripper, the first and second elongated medical members at respective first and second gripping points, wherein the first gripping point is a first portion of the first elongated medical member grippable by the first gripper and the second gripping point is a first portion of the second elongated medical member grippable by the second gripper;
moving, by a first and second mover, the first and second elongated medical members, wherein the first and second mover each comprise a pair of rollers, and wherein the first and second mover are configured to move the first and second elongated medical members via the respective pairs of rollers until the first elongated medical member is inserted into or extracted from within the second elongated medical member by a predetermined distance,
wherein, upon the insertion or extraction of the first elongated medical member into or from within the second elongated medical member by the predetermined distance, the first and second grippers grip the first and second elongated medical members at respective third and fourth gripping points, wherein the third gripping point is a second portion of the first elongated medical member grippable by the first gripper and the fourth gripping point is a second portion of the second elongated medical member grippable by the second gripper, wherein the first and second portions of each elongated medical member are different portions.

41. The method of claim 40, wherein the first gripper and the first mover are the same component, and wherein the second gripper and the second mover are the same component.

42. The method of claim 40, wherein said method is undertaken by a controller, and wherein the controller controls the gripping and moving steps of the method.

43-47. (canceled)

48. The medical apparatus of claim 1, wherein the first elongated medical member is configured to be inserted into and/or extracted from the second elongated medical member via inducing slack in the second elongated medical member.

49. The medical apparatus of claim 1, wherein the first elongated medical member is configured to be inserted into and/or extracted from the second elongated medical member manually by at least one user, and wherein the at least one user controls and/or performs one or more of the gripping, moving and releasing.

50. (canceled)

51. The medical apparatus of claim 1, wherein a distal tip of the second elongated medical member is configured to be kept in place during the insertion of the first elongated medical member into the second elongated medical member and/or the extraction of the first elongated medical member from the second elongated medical member.
